(19) United States
(12) Patent Application Publication
(10) Pub. No.: US 2026/0245716 A1
(43) Pub. Date: Aug. 20, 2026

(54) MMETHOD AND SYSTEM FOR INTERACTIVE ARTIFICIAL INTELLIGENCE (AI)-BASED PATIENT MONITORING

(71) Applicant: Cloudphysician Healthcare Pvt. Ltd., Bangalore (IN)

(72) Inventors: Dileep Raman, Bengaluru (IN); Dileep Unnikrishnan, Palakkad (IN); Dhruv Sud, Bengaluru (IN); Jitesh Sekar, Bengaluru (IN); Pradipkumar Maganbhai Solanki, Rajkot (IN)

(21) Appl. No.: 19/545,069

(22) Filed: Feb. 20, 2026

(30) Foreign Application Priority Data
Feb. 20, 2025 (IN) 202541014733

(51) Int. Cl. G16H 40/63; A61B 5/00

(52) U.S. Cl. CPC G16H 40/63; A61B 5/742; A61B 5/746; A61B 5/7475

(57) ABSTRACT

The present disclosure provides a method for interactive AI-based patient monitoring. The method includes receiving in real-time multimodal data of a patient through sensors. The multimodal data includes image data, video data, or audio data. The method includes splitting the multimodal data into chunks based on a predefined criterion, wherein each chunk is mapped to a corresponding time interval. The method includes assigning, via a multimodal model, an index tag to each chunk. The index tag includes a textual description of the associated chunk. The method includes determining, via a clinical decision model, clinical scores or attributes for each time interval based on keywords or visual patterns identified in the index tag or corresponding chunk. The method includes monitoring the clinical scores or predictions at each time interval based on predefined threshold scores or success criteria.

### DETAILED DESCRIPTION

### Technical Field

This disclosure generally relates to patient monitoring systems. More particularly, the disclosure relates to a method and a system for interactive Artificial Intelligence (AI)-based patient monitoring.

### Background

Existing patient monitoring technologies such as bedside monitors, wearable sensors, and electronic medical record (EMR) integrations have enabled the collection of real-time patient data across clinical and home settings. The systems track vital signs, activity levels, and medication use, and in some cases, trigger alerts when certain thresholds are crossed. Some platforms allow data sharing with clinicians or limited patient access through portals, and a few incorporate basic rule-based decision support.

However, the existing technologies often operate in isolation and may fail in providing meaningful insights upon interaction with the patient data collected from the various sensors. Additionally, the existing technologies generate alerts that may be prone to false alarms and may lack context.

There is, therefore, a requirement of a method for dynamically monitor patient vitals and interacting with care delivery partners for remedial actions.

### SUMMARY

In one embodiment, a method for interactive Artificial Intelligence (AI)-based patient monitoring is disclosed. The method includes receiving in real-time, by a processor, multimodal data of a patient through a set of sensors. The multimodal data includes at least one of image data, video data, or audio data. The method further includes splitting, by the processor, the multimodal data into a set of chunks based on a predefined criterion. Each of the set of chunks is mapped to a corresponding time interval of the multimodal data. The method further includes assigning, by the processor via a multimodal model, an index tag to each of the set of chunks. The index tag includes a textual description of a part or whole of an associated chunk. The method further includes, for each of the set of chunks, determining, by the processor via a clinical decision model, a set of clinical scores or attributes for the patient during the corresponding time interval based on one or more keywords or one or more visual patterns identified in the textual description of the index tag or in a corresponding chunk, respectively. The method further includes monitoring, by the processor via the clinical decision model, the set of clinical scores or predictions of the patient at each time interval based on a corresponding set of predefined threshold scores or success criteria.

In another embodiment, a system for interactive Artificial Intelligence (AI)-based patient monitoring is disclosed. The system includes a processor. The system further includes a memory communicatively coupled to the processor. The memory stores processor instructions, which when executed by the processor, cause the processor to receive in real-time, multimodal data of a patient through a set of sensors. The multimodal data includes at least one of image data, video data, or audio data. The processor instructions further cause the processor to split the multimodal data into a set of chunks based on a predefined criterion. Each of the set of chunks is mapped to a corresponding time interval of the multimodal data. The processor instructions further cause the processor to assign, via a multimodal model, an index tag to each of the set of chunks. The index tag includes a textual description of a part or whole of an associated chunk. The processor instructions further cause the processor to, for each of the set of chunks, determine, via a clinical decision model, a set of clinical scores or attributes for the patient during the corresponding time interval based on one or more keywords or one or more visual patterns identified in the textual description of the index tag or in a corresponding chunk, respectively. The processor instructions further cause the processor to monitor, via the clinical decision model, the set of clinical scores or predictions of the patient at each time interval based on a corresponding set of predefined threshold scores or success criteria.

In yet another embodiment, a non-transitory computer-readable medium storing computer-executable instructions for interactive Artificial Intelligence (AI)-based patient monitoring is disclosed. In one example, the stored instructions, when executed by a processor, may cause the processor to perform operations including receiving in real-time multimodal data of a patient through a set of sensors. The multimodal data includes at least one of image data, video data, or audio data. The operations further include splitting, by the processor, the multimodal data into a set of chunks based on a predefined criterion. Each of the set of chunks is mapped to a corresponding time interval of the multimodal data. The operations further include assigning, via a multimodal model, an index tag to each of the set of chunks. The index tag includes a textual description of a part or whole of an associated chunk. The operations further include, for each of the set of chunks, determining, via a clinical decision model, a set of clinical scores or attributes for the patient during the corresponding time interval based on one or more keywords or one or more visual patterns identified in the textual description of the index tag or in a corresponding chunk, respectively. The operations further include monitoring, via the clinical decision model, the set of clinical scores or predictions of the patient at each time interval based on a corresponding set of predefined threshold scores or success criteria.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this disclosure, illustrate exemplary embodiments and, together with the description, serve to explain the disclosed principles.

FIG. 1 is a block diagram of an exemplary system for interactive Artificial Intelligence (AI)-based patient monitoring, in accordance with some embodiments of the present disclosure.

FIG. 2 is a functional block diagram of an exemplary system for interactive AI-based patient monitoring, in accordance with some embodiments of the present disclosure.

FIG. 3 illustrates a schematic diagram of an exemplary system for object detection from live streams for contextual data retrieval, in accordance with some embodiments of the present disclosure.

FIG. 4 is a flow diagram of an exemplary method for interactive AI-based patient monitoring, in accordance with some embodiments of the present disclosure.

FIG. 5 is a flow diagram of an exemplary method for assigning index tags to chunks of multimodal data, in accordance with some embodiments of the present disclosure.

FIG. 6 is a flow diagram of an exemplary method for determining clinical scores or attributes of patients, in accordance with some embodiments of the present disclosure.

FIG. 7 illustrates a detailed flowchart of an exemplary method for interactive AI-based patient monitoring, in accordance with some embodiments of the present disclosure.

FIG. 8 illustrates a functional block diagram of an exemplary system for interactive AI-based patient monitoring and score calculation, in accordance with some embodiments of the present disclosure.

FIG. 9 illustrates an exemplary GUI for real-time video analysis and clinical action execution via multi-agent orchestration, in accordance with some embodiments of the present disclosure.

FIG. 10 illustrates an exemplary GUI for real-time video analysis and clinical action execution via multi-agent orchestration, in accordance with some embodiments of the present disclosure.

FIG. 11 illustrates an exemplary GUI for real-time video analysis and clinical action execution via multi-agent orchestration, in accordance with some embodiments of the present disclosure.

FIG. 12 illustrates an exemplary GUI for real-time video analysis and clinical action execution via multi-agent orchestration, in accordance with some embodiments of the present disclosure.

FIG. 13 is a block diagram of an exemplary computer system for implementing embodiments consistent with the present disclosure.

### DETAILED DESCRIPTION

Exemplary embodiments are described with reference to the accompanying drawings. Wherever convenient, the same reference numbers are used throughout the drawings to refer to the same or like parts. While examples and features of disclosed principles are described herein, modifications, adaptations, and other implementations are possible without departing from the spirit and scope of the disclosed embodiments. It is intended that the following detailed description be considered as exemplary only, with the true scope and spirit being indicated by the following claims.

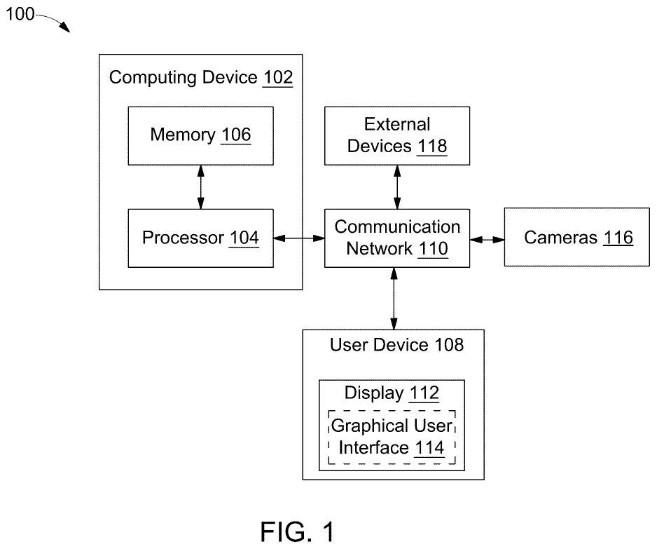

Referring now to FIG. 1, a block diagram of an exemplary system 100 for interactive Artificial Intelligence (AI)-based patient monitoring is illustrated, in accordance with some embodiments of the present disclosure. The system 100 may include a computing device 102, which, for example, may be, but is not limited to a server, a desktop, a laptop, a notebook, a netbook, a tablet, a smartphone, a mobile phone, or any other computing device. The computing device 102 may receive multimodal data for a patient as an input. Further, the computing device 102 may generate a combined response based on the response of one or more of a plurality of AI agents.

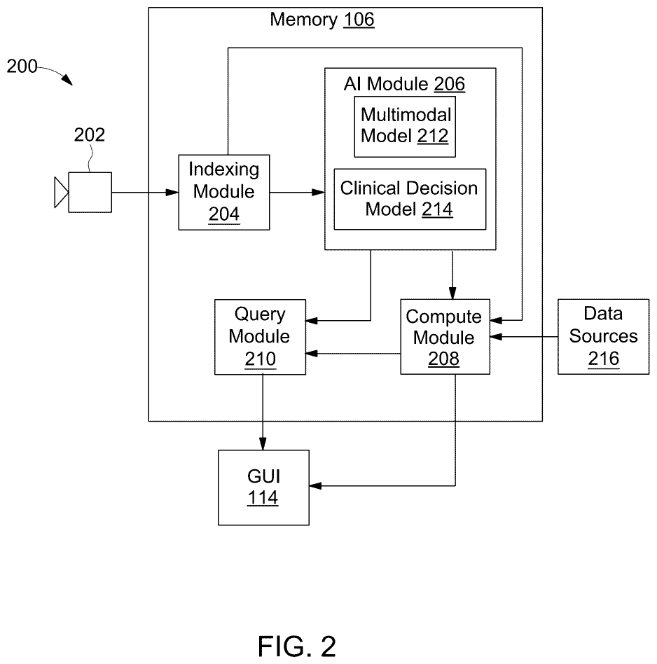

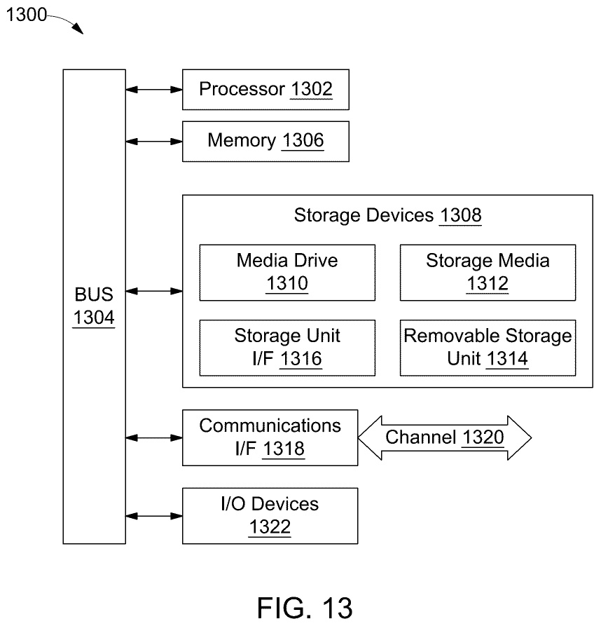

As will be described in greater detail in conjunction with FIGS. 2-13, the computing device 102 may receive in real-time, multimodal data of a patient through a set of sensors. The multimodal data may include at least one of image data, video data, or audio data. Further, the computing device 102 may split the multimodal data into a set of chunks based on a predefined criterion. Each of the set of chunks may be mapped to a corresponding time interval of the multimodal data. Further, the computing device 102 may assign, via a multimodal model, an index tag to each of the set of chunks. The index tag may include a textual description of a part of or whole of an associated chunk. Further, for each of the set of chunks, the computing device 102 may determine, via a clinical decision model, a set of clinical scores or attributes for the patient during the corresponding time interval based on one or more keywords or one or more visual patterns identified in the textual description of the index tag or in a corresponding chunk, respectively. Further, the computing device 102 may monitor, via the clinical decision model, the set of clinical scores or predictions of the patient at each time interval based on a corresponding set of predefined threshold scores or success criteria.

Further, the computing device 102 may include a processor 104 and a memory 106. In one embodiment, the computing resource may be the processor 104. The memory 106 may store instructions that, when executed by the processor 104, cause the processor 104 to monitor patients virtually, in accordance with aspects of the present disclosure. The memory 106 may also store various data (for example, multimodal data, a real-time video, a plurality of frames, a plurality of index tags, a combined response, a predefined threshold, a prompt, a patient summary, an EMR, a medical knowledge base and the like) that may be captured, processed, and/or required by the system 100.

The system 100 may further include a user device 108 communicatively connected to the computing device 102 over a communication network 110 for sending or receiving various data. By way of an example, the user device 108 may be, but may not be limited to, a smartphone, a tablet, a laptop, a netbook, a notebook, or any other computing device. The user device 108 may include a display 112. A user may interact with the computing device 102 via a GUI 114 accessible via the display 112. The system 100 may also include one or more cameras 116, each communicatively connected to the computing device 102 through the communication network 110. The one or more cameras 116 may be deployed in a room with a patient. The one or more cameras 116 may be configured to capture and/or record the real-time video of the patient.

Further, the system 100 may include one or more external devices 118. The computing device 102 may interact with the one or more external devices 118 over the communication network 110 for sending or receiving various data. The communication network 110, for example, may include, but may not be limited to, a Wireless Fidelity (Wi-Fi) network, a Light Fidelity (Li-Fi) network, a Local Area Network (LAN), a Wide Area Network (WAN), a Metropolitan Area Network (MAN), a satellite network, the internet, a fiber optic network, a coaxial cable network, an infrared (IR) network, a Radio Frequency (RF) network, or a combination thereof. The one or more external devices 118 may include, but may not be limited to a remote server, a laptop, a netbook, a notebook, a smartphone, a mobile phone, a tablet, or any other computing device.

Referring now to FIG. 2, a functional block diagram of an exemplary system 200 configured for interactive AI-based patient monitoring is illustrated, in accordance with some embodiments of the present disclosure. FIG. 2 is explained in conjunction with FIG. 1. The system 200 may be analogous to the system 100. The system 200 may include a camera 202 (analogous to the one or more cameras 116). The system 200 may further include, within the memory 106 of the computing device 102, an indexing module 204, an AI module 206, a compute module 208, and a query module 210. The AI module 208 may include a multimodal model 212 and a clinical decision model 214.

By way of an example, the multimodal model 212 may be, but may not be limited to, a multimodal AI model with transformer architecture (for example, a Vision Model). The clinical decision model 214 may be, but may not be limited to, a reasoning model of transformer architecture, a Large Language Model (LLM), a decision tree-based model, a random forest model, a gradient boosting model, a neural network-based clinical decision support model, a Bayesian network model, a rule-based expert system, or any combination thereof. In some embodiments, one or both of the multimodal model 212 and the clinical decision model 214 may be hosted on external servers.

The system 200 may further include one or more data sources 216 (for example, a patient Electronic Medical Record (EMR) database, device databases, environment databases, etc.). It should be noted that the one or more data sources 216 may be hosted on external devices (for example, external servers).

The camera 202 may capture a real-time video of a patient. The camera 202 may then send the real-time video to the indexing module 204. The real-time video may include a plurality of frames. Additionally, the indexing module 204 may receive images or audio streaming from additional monitoring devices (additional cameras, microphones, etc.). For example, a microphone may be placed around the bed of the patient to monitor patient behaviour or any inputs discussed by a doctor or a nurse near the bed. Thus, the indexing module 204 may receive multimodal data related to the patient in real-time from a set of sensors (such as the camera 202). Further, the indexing module 204 may split the multimodal data into a set of chunks based on a predefined criterion. Each of the set of chunks may be mapped to a corresponding time interval of the multimodal data to ensure that the multimodal data is synchronized. The predefined criterion may correspond to one of a predefined time interval or a predetermined content portion.

The indexing module 204 may then send the set of chunks to the AI module 206. Further, the AI module 206 may assign, via the multimodal model 212, an index tag to each of the set of chunks. The index tag may include a textual description of a part or whole of an associated chunk. To assign the index tag to each chunk of the set of chunks, the AI module 206 may extract, via the multimodal model 212, relevant clinical information from the chunk. Further, the AI module 206 may generate, via the multimodal model 212, the textual description corresponding to the chunk. Further, the AI module 206 may create, via the multimodal model 212, the index tag for the chunk based on the clinical information and the textual description. The AI module 206 may then send the set of chunks and an associated set of index tags to the indexing module 204. The indexing module 204 may store a mapping of the set of chunks and the associated set of index tags in a vector database (not shown).

The compute module 208 may retrieve the set of chunks and the associated set of index tags from the vector database. Additionally, the compute module 208 may retrieve patient contextual data from the one or more data sources 216. Further, the compute module 208 may send the set of chunks, the associated set of index tags, and the patient contextual data to the AI module 206. For each of the set of chunks, the AI module 206 may determine, via the clinical decision model 214 and using the patient contextual data, a set of clinical scores or attributes for the patient during the corresponding time interval based on one or more keywords or one or more visual patterns identified in the textual description of the index tag or in a corresponding chunk, respectively. In other words, the clinical decision model 214 may identify textual patterns (i.e., the one or more keywords) from the textual description of the index tag. Additionally or alternatively, the clinical decision model 214 may identify the one or more visual patterns from the chunk of the multimodal data. Using the textual patterns and/or the visual patterns, the clinical decision model 214 may then determine the set of clinical scores or attributes of the patient. By way of example, the set of clinical scores may include, but is not limited to, a Richmond Agitation-Sedation Scale (RASS) score, a Glasgow Coma Scale (GCS) score, a pain assessment score, a Sequential Organ Failure Assessment (SOFA) score, a mobility score, a fall risk score, a pressure injury risk score, or any combination thereof. By way of example, the set of attributes may include, but is not limited to, patient posture, eye opening status, movement patterns, facial expressions indicative of pain or distress, presence of medical devices or tubes, patient responsiveness to stimuli, or any combination thereof. Further, the AI module 206 may send the set of clinical scores or attributes of the patient to the compute module 208.

The system 200 may further include a user device (such as the user device 108) in communication with the computing device 102. The GUI 114 may be rendered on the display 112 of the user device 108. The GUI 114 may present the real-time video of the patient along with various patient health parameters (e.g., vitals) and the set of clinical scores or attributes. The compute module 208 may monitor the set of clinical scores or predictions of the patient at each time interval based on a corresponding set of predefined threshold scores or success criteria using the clinical decision model 214 of the AI module 206. By way of example, the predictions may include, but are not limited to, risk of patient deterioration, likelihood of sepsis onset, probability of respiratory failure, predicted need for mechanical ventilation, anticipated length of ICU stay, risk of cardiac arrest, likelihood of patient fall, or any combination thereof. To monitor the set of clinical scores or predictions of the patient, for each clinical score of the set of clinical scores or attributes, the clinical decision model 214 may compare the clinical score with a corresponding predefined threshold score. When the clinical score is beyond the predefined threshold score (for example, less than a lower limit score or greater than an upper limit score), the compute module 208 may render, via the GUI 114, an alert corresponding to the patient based on the comparing. The alert may be provided in a textual, visual, or an audio format to notify a health provider of a potentially deteriorating patient health condition.

The GUI 114 may also provide a GUI element for a user to input a user query related to the patient to seek a clinical practice recommendation for the patient. The user query may be in text, audio, or video format. Once the user provides the user query through the GUI 114, the user query is sent to the query module 210 of the computing device 102. The query module 210 may send the user query to the AI module 206. The clinical decision model 214 may then generate a clinical practice recommendation corresponding to the patient based on the monitoring of the set of clinical scores or attributes in response to the user query. The query module 210 may then render, via the GUI 114, the clinical practice recommendation corresponding to the patient. The clinical practice recommendation may be overlaid on the real-time video of the patient in the GUI 114. In some embodiments, the compute module 208 may autonomously prompt the clinical decision model 214 to generate the clinical practice recommendation corresponding to the patient based on the monitoring of the set of clinical scores or attributes relative to the predefined threshold scores. In such embodiments, the compute module 208 may render, via the GUI 114, the clinical practice recommendation generated by the clinical decision model 214.

By way of an example, Table 1 below provides an exemplary list of

| Clinical | | | | Frequency |
|---|---|---|---|---|
| Score/ | | | | of |
| Attribute | Disease | Domain | Desired outcome | Monitoring |
| Visual | Delirium | PAD | Sort patients into agitated (needing | Every hour |
| RASS | | | sedation), calm (adequate sedation), | |
| | | | unresponsive (over sedated). Warn | |
| | | | the nurse that the patient is over | |
| | | | sedated and that the visual RASS | |
| | | | score is X, and that a more formal and | |
| | | | accurate RASS is needed. Also serve | |
| | | | as a trigger to reduce sedation that | |
| | | | may reduce LOS. | |
| Visual | NA | PAD | Detect visual pain scores and alert | Every hour |
| CPOT | | | nurses on whether the patient needs | |
| | | | more pain meds or the patient is | |
| | | | getting too much pain medication | |
| | | | (very low scores). Combined with | |
| | | | RASS it will give the overall level of | |
| | | | distress for the patient. This distress is | |
| | | | the non respiratory kind. Pull drug list | |
| | | | of sedatives and pain meds from | |
| | | | RADAR on video UI as an adjunct UI | |
| | | | input (also applies to vRASS). | |
| Respiratory | ARDS | vNurse | Detect respiratory rate and work of | Every 3 |
| distress | | | breathing. Rate must be from direct | minutes |
| | | | video assessment and then correlated | |
| | | | with the monitor and not directly from | |
| | | | the monitor. WOB should be | |
| | | | described by looking at neck, chest | |
| | | | and abdominal movements. Sweating, | |
| | | | eye movements, gasping, eye | |
| | | | opening (staring looks showing agony, | |
| | | | fear), forehead frowning and | |
| | | | grimacing, tripod arm and body | |
| | | | position and attachment of respiratory | |
| | | | devices or oxygen. Pull current vent or | |
| | | | fio2 settings from RADAR on the video | |
| | | | UI. | |
| Position | Pressure | vNurse | Detect patient body position (supine, | Every 90 |
| (skin | sore | | prone, left lateral, right lateral) and | minutes |
| health) | | | then autochart it. Also send an alert to | |
| | | | the nurse if these positions have not | |
| | | | changed for 90 minutes (yellow alert), | |
| | | | 120 minutes (red alert) | |
| Change in | Delirium | vNurse | Detect if there is a sudden change in | Every 3 |
| mental | | | mental status for a patient who is in | minutes |
| status | | | respiratory distress | |
| Seizures | Seizures | Neuro | Detect abnormal high frequency | Every 5 |
| | | | movements in arms, legs or face | seconds |
| | | | (twitches) that are consistent with | |
| | | | seizures. | |
| Call for | NA | vSitter | Detect when the patient is calling for | Every 3 |
| help | | | assistance either visually or with | seconds |
| | | | audio. | |
| CPR in | Resuscitation | Safety | Tell the vNurse that multiple people | Every 10 |
| progress | | | have gathered around the patient and | seconds |
| | | | that a CPR of rounds event is in | |
| | | | progress | |
| CLABSI | Quality | Infection | After HH steps, check if the hub was | Every |
| | | control | cleaned before use. Check the ‘Scrub | instance of |
| | | | the Hub’ image. Make sure that the | hub |
| | | | proceduralist was wearing gowns | interaction |
| | | | during insertion, make sure the field | and C line |
| | | | was prepared during insertion. | insertion. |
| Height | ARDS | vNurse | Measure the height of the patient on | Once per |
| measurement | | | demand | admission |
| Falls | NA | vSitter | Detect risk of fall before the fall | Every 5 |
| | | | happens and alert the bedside in time | seconds |
| | | | so that they can reposition the patient | |
| | | | or come to the bedside and evaluate. | |
| Hand | Quality | Infection | Detect is the nurse or physician has | Every |
| hygiene | | control | used either gloves or hand rub prior to | interaction |
| | | | a patient interaction. Judge the quality | |
| | | | of hand hygiene based on steps of HH. | |
| | | | Special focus on if the event was | |
| | | | touching IV lines, tubes and catheters | |
| | | | connected to the patient. | |
| On bed off | NA | vSitter | Alert the bedside if the patient is | Every 10 |
| bed | | | ‘missing’ or if a bed is now occupied | minutes |
| | | | (and it was not before) | |
| ET or | ARDS | vNurse | Detect if the ventilator is disconnected | Every 5 |
| Trach tube | | | from the patient (either ETT or | seconds |
| disconnection | | | tracheostomy tube) | |
| Curtain | NA | vSitter | Detect if the bed is covered by curtains | Every 10 |
| over | | | and send an alert to the vNurse that a | seconds |
| bedside | | | private event for the patient may have | |
| | | | started. | |
| VAP | ARDS | Infection | Detect if the head of the bed is at a 30 | Every 15 |
| | | control | deg angle, ensure closed suction is in | minutes |
| | | | place, check if HME or tubing is soiled. | |
| | | | Is oral care happening at least 2 times | |
| | | | per day, does the patient have a tube | |
| | | | with subglottic suction (identify tube | |
| | | | visually), check with RADAR is there is | |
| | | | a drug for ulcer prophylaxis (show on | |
| | | | the video UI as an input from RADAR), | |
| | | | check if someone is changing the | |
| | | | ventilator to a spontaneous mode | |
| | | | once a day, with a high RASS (0 or −1), | |
| | | | ensure HH is performed each time | |
| | | | the circuit is touched, has the cuff | |
| | | | pressure been measured at least | |
| | | | every 4 hours (see YT video on this). | |
| Dysphagia | Pneumonia | Neuro | Detect risk factors for dysphagia and | Triggered |
| | (Aspiration) | | predict aspiration risk | by feeding |
| | | | | event |
| Auto GCS | TBI/Neuro | Neuro | Automatically compute Glasgow | Case-to- |
| | injury | | Coma Scale via visual, motor, verbal | case basis |
| | | | cues. | |
| Auto NIH | Stroke | Neuro | Measured every 15 min, then hourly. | Case-to- |
| stroke | | | Huge documentation burden for | case basis |
| scale - | | | nurses. | |
| prompted | | | | |
| C-diff | Infection | Infection | Detect isolation compliance, PPE use, | Case-to- |
| | | | stool frequency patterns, and cleaning | case basis |
| | | Control | compliance. | |
| Admission | NA | Workflow/ | Detect and document completion of | On |
| | | Quality | admission workflow steps including | admission |
| | | | patient identification band placement, | |
| | | | initial positioning, device attachment | |
| | | | (monitor, oxygen, IV), baseline mental | |
| | | | status capture, and room readiness | |
| | | | verification. Generate alert if | |
| | | | admission protocol steps are | |
| | | | incomplete. | |
| Delirium | Delirium | Neuro | Emotion, motion and pose. Ref CAM- | Case-to- |
| | | | ICU 7. Look at the face, lips, eyes. | case basis |
| SCD | DVT | Quality | Detect whether the machine on or off | Case-to- |
| compliance | prevention | | the legs of the patient. | case basis |
| Bipap on | ARDS | vNurse | Detect oxygen delivery device type | Case-to- |
| VS off, | | | and whether device is on/off. | case basis |
| NRB on vs | | | | |
| off, | | | | |
| cannula on | | | | |
| vs off | | | | |
| Body | Pressure | vNurse | Detect approximate bed angle and | Case-to- |
| position | sore/ARDS | | patient torso elevation. | case basis |
| with rough | | | | |
| angle | | | | |
| Patient | NA | vSitter | Measure time from patient call to | Case-to- |
| response | | | bedside response. | case basis |
| times (call | | | | |
| for help) | | | | |
| Device | ICU | vNurse | Determine what devices are at the | Case-to- |
| recognition | workflow | | bedside - monitor, vent, dialysis, SCD, | case basis |
| and auto | | | IV pole and pump. | |
| presets | | | | |
| Vent | ARDS | vNurse | Analyze ventilator screen parameters | Case-to- |
| screen | | | visually and correlate with patient | case basis |
| analysis | | | condition. | |

It should be noted that all such aforementioned modules 204-210 may be represented as a single module or a combination of different modules. Further, as will be appreciated by those skilled in the art, each of the modules 204-210 may reside, in whole or in parts, on one device or multiple devices in communication with each other. In some embodiments, each of the modules 204-210 may be implemented as dedicated hardware circuit comprising custom application-specific integrated circuit (ASIC) or gate arrays, off-the-shelf semiconductors such as logic chips, transistors, or other discrete components. Each of the modules 204-210 may also be implemented in a programmable hardware device such as a field programmable gate array (FPGA), programmable array logic, programmable logic device, and so forth. Alternatively, each of the modules 204-210 may be implemented in software for execution by various types of processors (e.g., the processor 104). An identified module of executable code may, for instance, include one or more physical or logical blocks of computer instructions, which may, for instance, be organized as an object, procedure, function, or other construct. Nevertheless, the executables of an identified module or component need not be physically located together but may include disparate instructions stored in different locations which, when joined logically together, include the module, and achieve the stated purpose of the module. Indeed, a module of executable code could be a single instruction, or many instructions, and may even be distributed over several different code segments, among different applications, and across several memory devices.

As will be appreciated by one skilled in the art, a variety of processes may be employed for interactive AI-based patient monitoring. In particular, as will be appreciated by those of ordinary skill in the art, control logic and/or automated routines for performing the techniques and steps described herein may be implemented by the system 100 and the associated computing device 102, either by hardware, software, or combinations of hardware and software. For example, suitable code may be accessed and executed by the one or more processors on the system 100 to perform some or all of the techniques described herein. Similarly, application specific integrated circuits (ASICs) configured to perform some, or all of the processes described herein may be included in the one or more processors on the system 100.

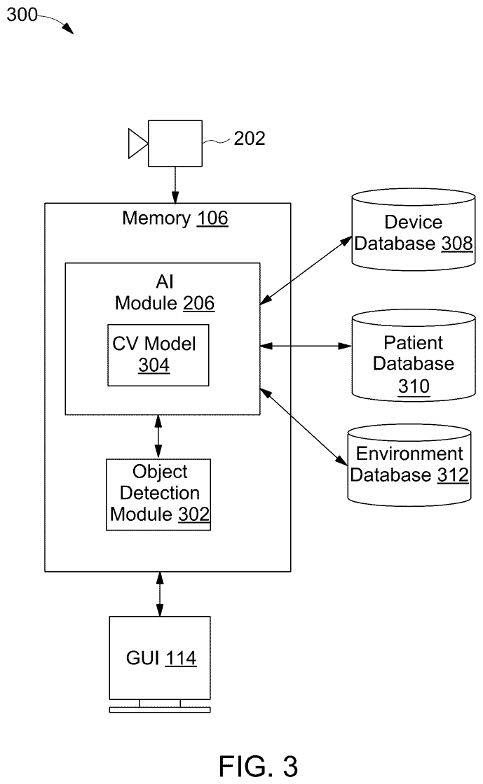

Referring now to FIG. 3, a schematic diagram of an exemplary system 300 for object detection from live streams for contextual data retrieval is illustrated, in accordance with some embodiments of the present disclosure. FIG. 3 is explained in conjunction with FIGS. 1 and 2. In an embodiment, the system 300 may be deployed in combination with the system 200. It should be noted that in such a deployment, the combination of the system 200 and the system 300 may be analogous to the system 100 of FIG. 1. The system 300 may include the camera 202 communicably coupled to the computing device 102. The computing device 102 may include, within the memory 106, the AI module 206 and an object detection module 302. The AI module 206 may include a CV model 304. It should be noted that the AI module 206 may also include the multimodal model 212 and the clinical decision model 214 as explained in conjunction with FIG. 2. Further, the system 300 may include a device database 308, a patient database 310, and an environment database 312. It should be noted that the device database 308, the patient database 310, and the environment database 312 are included in the one or more data sources 216.

Further, the computing device 102 may be communicably coupled to the user device 108. The user device 108 may render the GUI 114 on the display 112. The camera 202 may capture the real-time video of the patient. Further, the object detection module 302 may receive the real-time video from the camera 202. The object detection module 302 may send the real-time video to the AI module 206. Further, the AI module 206 may identify, using the CV model 304, one or more objects around the patient in each of the plurality of frames of the real-time video. By way of an example, the CV model 304 may be based on a Convolutional Neural Network (CNN) architecture. The one or more objects may include, but are not limited to, face of the patient, body and behaviour of the patient, screens of various patient health monitoring devices (such as a cardiac monitor, a ventilator screen, etc.), tubes and bags attached to the patient (e.g., urine collection bag, IV fluid bag, etc.) or the like.

For each object of the one or more objects, the CV model 304 may determine an area of coverage of the object in the frame. When the area of coverage may be above a predefined threshold, the AI module 206 may retrieve relevant data (at least one of relevant patient data, device data, or environment data) associated with the object from one or more of the data sources 216 (i.e., from one or more of the device database 308, the patient database 310, and the environment database 312). The predefined threshold may be any predetermined proportion of the frame. In one example, the predefined threshold may be 60% area of coverage of the frame. When an object is detected with the coverage of the frame above the predefined threshold, the relevant data may be retrieved by the AI module 206. The relevant data may then be processed by the CV model 304 and/or the clinical decision model 214. The output obtained from the processing of the relevant data may then be rendered on the user device 304 via the GUI 114.

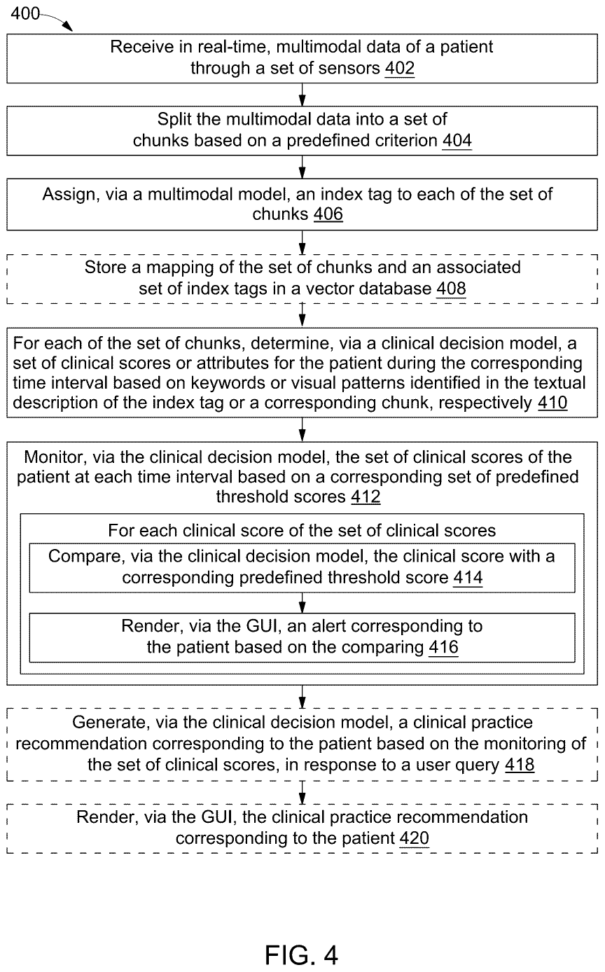

Referring now to FIG. 4, a flow diagram of an exemplary method 400 for interactive AI-based patient monitoring is illustrated, in accordance with some embodiments of the present disclosure. FIG. 4 is explained in conjunction with FIGS. 1-3. The method 400 may be implemented by the computing device 102 of the system 100. The method 400 may include receiving in real-time, by a processor (such as the processor 104), multimodal data of a patient through a set of sensors, at step 402. The multimodal data may include at least one of image data, video data, or audio data. Further, the method 400 may include splitting, by the processor, the multimodal data into a set of chunks based on a predefined criterion, at step 404. Each of the set of chunks may be mapped to a corresponding time interval of the multimodal data. Further, the method 400 may include assigning, by the processor via a multimodal model (such as the multimodal model 212), an index tag to each of the set of chunks, at step 406. The index tag may include a textual description of a part or whole of an associated chunk. Further, the method 400 may include storing a mapping of the set of chunks and an associated set of index tags in a vector database, at step 408. It should be noted that step 408 is an optional step in the method 400.

Further, the method 400 may include, for each of the set of chunks, determining, by the processor via a clinical decision model (such as the clinical decision model 214), a set of clinical scores or attributes for the patient during the corresponding time interval based on one or more keywords or one or more visual patterns identified in the textual description of the index tag or in a corresponding chunk, respectively, at step 410. Further, the method 400 may include monitoring, by the processor via the clinical decision model, the set of clinical scores or predictions of the patient at each time interval based on a corresponding set of predefined threshold scores or success criteria, at step 412. Further, the method 400 may include, for each clinical score of the set of clinical scores or attributes, comparing, via the clinical decision model, the clinical score with a corresponding predefined threshold score, at step 414. Further, the method 400 may include rendering, via a GUI (such as the GUI 114), an alert corresponding to the patient based on the comparing, at step 416. Further, the method 400 may include generating, via the clinical decision model, a clinical practice recommendation corresponding to the patient based on the monitoring of the set of clinical scores or attributes, in response to a user query, at step 418. Further, the method 400 may include rendering, via the GUI, the clinical practice recommendation corresponding to the patient, at step 420. The clinical practice recommendation may be overlaid on a real-time video of the patient in the GUI. It should be noted that steps 418 and 420 are optional steps in the method 400.

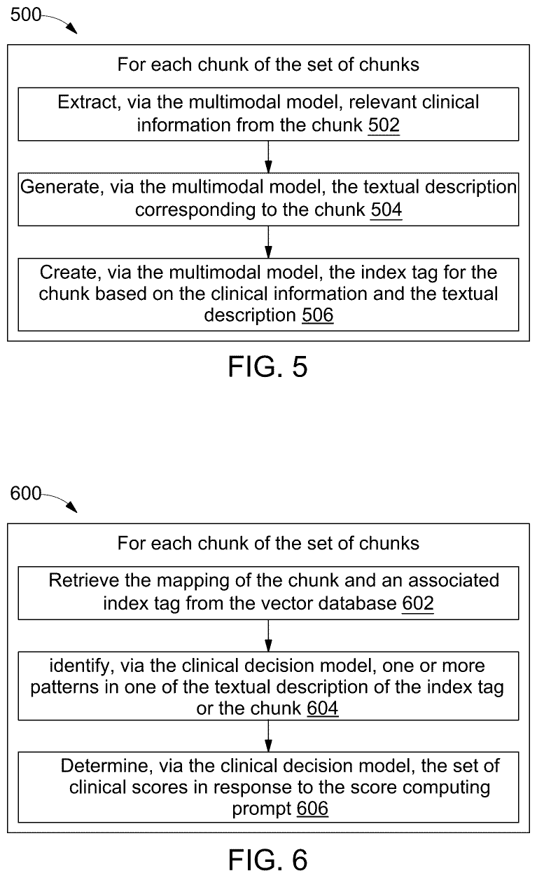

Referring now to FIG. 5, a flow diagram of an exemplary method 500 for assigning index tags to chunks of multimodal data is illustrated, in accordance with some embodiments of the present disclosure. FIG. 5 is explained in conjunction with FIGS. 1-4. The method 500 may be performed for each chunk of the set of chunks. The method 500 may include extracting, via the multimodal model, relevant clinical information from the chunk, at step 502. Further, the method 500 may include generating, via the multimodal model, the textual description corresponding to the chunk, at step 504. Further, the method 500 may include creating, via the multimodal model, the index tag for the chunk based on the clinical information and the textual description, at step 506.

Referring now to FIG. 6, a flow diagram of an exemplary method 600 for determining clinical scores or attributes of patients is illustrated, in accordance with some embodiments of the present disclosure. FIG. 6 is explained in conjunction with FIGS. 1-5. The method 600 may be performed for each chunk of the set of chunks. The method 600 may include retrieving the mapping of the chunk and an associated index tag from the vector database, at step 602. Further, the method 600 may include identifying, via the clinical decision model, one or more patterns in one of the textual description of the index tag or the chunk, at step 604. The one or more patterns may include one or more keywords in the textual description of the index tag or one or more visual patterns in the chunk. Further, the method 600 may include determining, via the clinical decision model, the set of clinical scores or attributes based on the one or more patterns, at step 606.

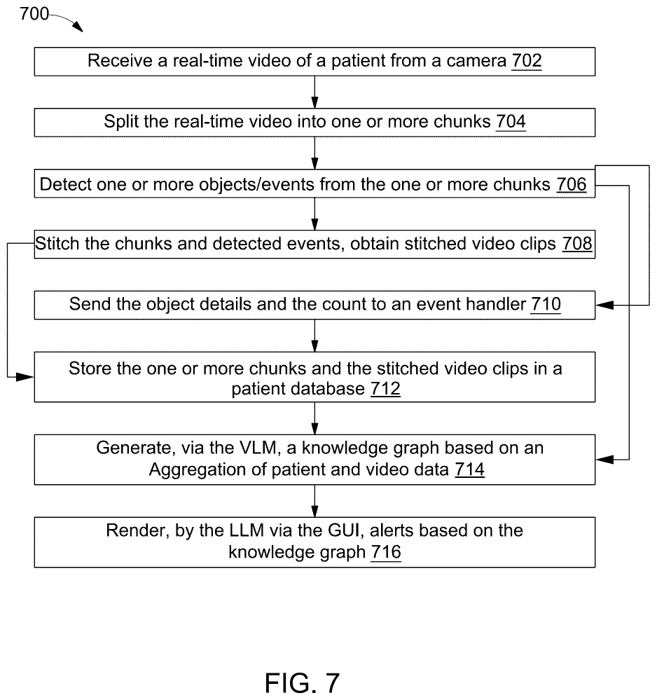

Referring now to FIG. 7, a detailed flowchart of an exemplary method 700 for interactive AI-based patient monitoring is illustrated, in accordance with some embodiments of the present disclosure. FIG. 7 is explained in conjunction with FIGS. 1-7. The method 700 may be implemented by the computing device 102 of the system 100. The method 700 may include receiving a real-time video of a patient from a camera (such as the camera 202), at step 702. The real-time video may include a plurality of frames. Further, the method 700 may include splitting the real-time video (and other multimodal data) into one or more chunks, at step 704. A chunk may include one or more of the plurality of frames in case of the real-time video. Additionally, after step 704, the method 700 may include detecting one or more objects/events from the one or more chunks, at step 706. The object/event details and parameters associated with the objects detected may be stored in a timeseries database. For example, the parameters may include respiratory rate detected on a ventilator screen detected in a frame. In some embodiments, the timeseries database may store timestamp corresponding to the one or more objects in the real-time video. The timeseries database may help in detecting the exact time when an undesired event happened. In one example, if the heart rate may increase or decrease beyond a predefined standard value that timestamp may be determined from the timeseries database and the corresponding events triggered due to the change may be noted to determine remedial actions. The remedial actions may include but are not limited to increase or decrease sedation, administer pain medications and escalate antibiotics.

Further, the method 700 may include stitching the one or more chunks and the detected objects/events to obtain stitched video clips, at step 708. Further, the method 700 may include sending the object/event details and the parameters to an event handler, at step 710. Further, the method 700 may include storing, via the event handler, the one or more chunks and the stitched video clips in a patient database based on events, at step 712. Further, the method 700 may include sending the detected events and the one or more chunks to a multimodal model (for example, a Vision Language Model (VLM) or any other multimodal AI model with a transformer architecture). Further, the method 700 may include generating, via the VLM, a knowledge graph based on an aggregation of patient and video data, at step 714. The aggregated data may include detected events, object details, count of objects, patient's EMR data, the one or more chunks and the like.

Further, the method 700 may include calculating, via the clinical decision model, a set of clinical scores and attributes for the patient using the knowledge graph. Further, the method 700 may include rendering, by an LLM via the GUI (for example, the GUI 114), alerts based on the set of clinical scores and attributes and the knowledge graph, at step 716. The alerts may be overlaid on the real-time video of the patient in the GUI.

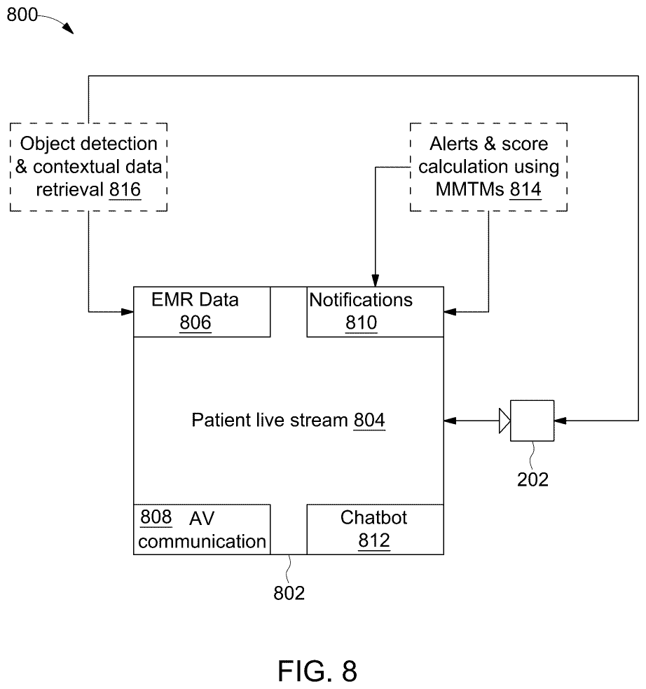

Referring now to FIG. 8, a functional block diagram of an exemplary system 800 for interactive AI-based patient monitoring is illustrated, in accordance with some embodiments of the present disclosure. FIG. 8 is explained in conjunction with FIGS. 1-6. The system 800 may include a camera 202. The camera 202 may be configured to capture a real-time video of the patient. Further, the system 800 may include the user device 108 (not shown). The user device 108 may render a GUI 702 (analogous to the GUI 114) on the display 112. The GUI 802 may present the real-time video as a patient live stream 704. Additionally, the GUI 802 may include auxiliary data and GUI elements overlaid upon the real-time video. For example, the auxiliary data and GUI elements may include EMR data 806 overlaid at top left corner, an AV communication element 808 overlaid at bottom left corner, notifications 810 overlaid at right corner, and a chatbot 812 overlaid at bottom right corner.

The system 800 may also include the computing device 102 (not shown) communicably coupled to the camera 202 and the user device 108. The computing device 102 may implement an alerts and score calculation component 814 and an object detection and contextual data retrieval component 816. It should be noted that the system 800 may be analogous to the system 200 and the system 300. It should also be noted that the implementation of the alerts and score calculation component 714 and the object detection and contextual data retrieval component 816 may be facilitated by equivalent modules defined in the system 200 and the system 300 in conjunction with FIG. 2 and FIG. 3, respectively.

The camera 202 may capture the patient live stream 804 and may transmit the patient live stream 804 to the object detection and contextual data retrieval component 816. The object detection and contextual data retrieval component 816 may perform object detection and contextual data retrieval functions on the incoming video stream. This has already been explained in detail in conjunction with FIG. 3.

The alerts and score calculation component 714 may receive the multimodal data from (such as the patient live stream 804 from the camera 202) and may process each chunk of the multimodal data to generate textual descriptions, assign index tags to each frame based on the generated textual descriptions, and store the indexed chunks along with the associated index tags in the databases 214. The indexed chunks and index tags may be subsequently retrieved by the clinical decision model 214 when calculating the set of clinical scores or attributes. This has already been explained in detail in conjunction with FIG. 2.

The chatbot 812 may provide an interface for a user to input queries related to the patient. Upon receiving a user query through the chatbot 812, the computing device 102 may determine one or more of the agents 212 to process the query based on the query content, the indexed frames, and the associated index tags. The agents 212 may generate individual agent responses, which may be combined to form a combined response. The combined response may be rendered via the chatbot 812 and may be overlaid on the patient live stream 804 in the GUI 802.

The EMR data 806 may display patient health parameters retrieved from the databases 214, including vital signs, laboratory values, and medication information. The notifications 810 may display alerts and patient updates generated based on detected events in the patient live stream 804. The AV communication element 808 may enable audio and video communication between the user and other healthcare personnel or the patient, facilitating remote consultation and care coordination.

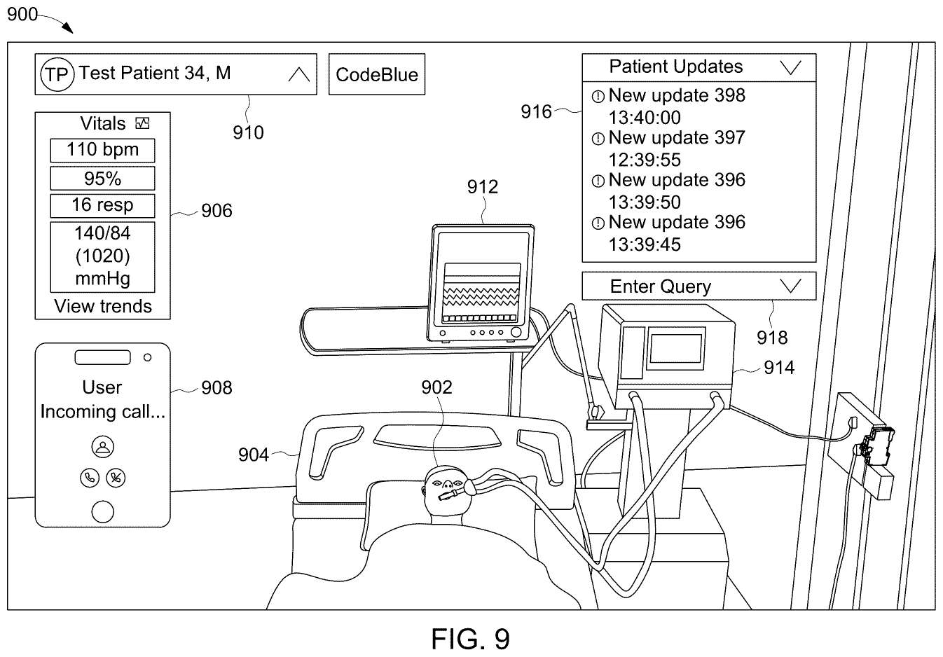

Referring now to FIG. 9, an exemplary GUI 900 for interactive AI-based patient monitoring is illustrated, in accordance with some embodiments of the present disclosure. FIG. 9 is explained in conjunction with FIGS. 1-8. The GUI 900 may display a real-time video feed of a patient 902 lying on a hospital bed 904 in a clinical setting. A vitals panel 906 may be positioned on the upper left corner of the GUI 900 and may display patient vital signs including heart rate, oxygen saturation, respiratory rate, and blood pressure readings. A communication interface 908 may be located on the lower left corner of the GUI 900 and may show an incoming call notification with options to accept or decline the call. A patient information bar 910 may be positioned at the top centre of the GUI 900 and may display patient identification information including initials, name, age, and gender, along with a status indicator. A patient monitor 912 may be visible in the background of the real-time video feed mounted on an adjustable arm near the hospital bed 904. A ventilator screen 914 may be positioned adjacent to the patient monitor 912 and may display ventilator-related information. A notification center 916 may be located on the upper right corner of the GUI 900 and may display a list of patient updates with timestamps. An AI chat interface 918 may be positioned below the notification center 916 and may provide an interface for multimodal interaction with the query module 210 and the compute module 208.

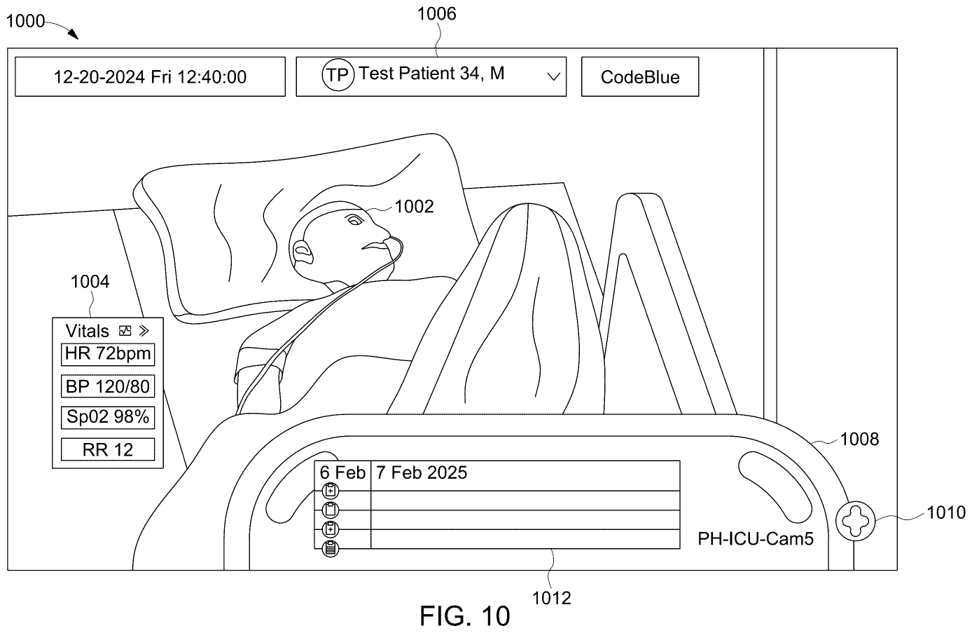

Referring now to FIG. 10, a GUI 1000 for interactive AI-based patient monitoring is illustrated, in accordance with some embodiments of the present disclosure. FIG. 10 is explained in conjunction with FIGS. 1-8. The GUI 1000 may display a real-time video 1002 of a patient lying in a hospital bed as the background of the interface. A vitals panel 1004 may be positioned on the left side of the GUI 1000 and may display patient vital signs including heart rate, blood pressure, SpO2, and respiratory rate. A patient information display 1006 may be located at the top center of the GUI 1000 and may show patient identification information including the patient name and a status indicator. Bedside equipment 1008 may be visible on the right side of the real-time video 1002 and may include items (such as beverage containers and medical supplies on a bedside table). An AI interaction interface 1010 may be positioned in the lower right portion of the GUI 1000 and may provide a mechanism for multimodal interaction with a reasoning engine. A timeline interface 1012 may be displayed at the bottom of the GUI 1000 and may show a chronological view of patient events with date markers and event indicators, allowing navigation through recorded patient data and video streams.

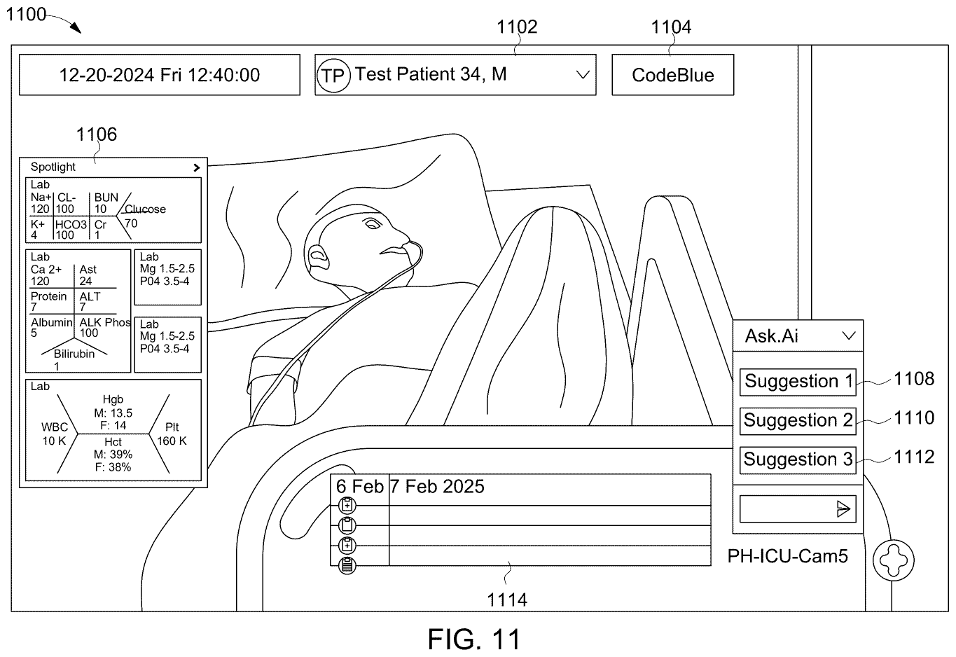

Referring now to FIG. 11, a GUI 1100 for interactive AI-based patient monitoring is illustrated, in accordance with some embodiments of the present disclosure. FIG. 11 is explained in conjunction with FIGS. 1-10. The GUI 1100 may display a real-time video feed of a patient in a hospital bed as the background, with the patient shown wearing medical equipment including nasal tubing. A patient information bar 1102 at the top centre of the GUI 1100 may display patient identification information (for example, name of the patient is “Abhinab Barman”) along with an alert button 1104. On the left side of the GUI 1100, a spotlight panel 1106 may display laboratory values and vital signs data organized in multiple sections showing values for parameters such as glucose, potassium, HCO3, creatinine, magnesium, phosphate, WBC count, haemoglobin, haematocrit, and platelet count. On the right side of the GUI 1100, an AI interaction interface may present a first suggestion 1108, a second suggestion 1110, and a third suggestion 1112 as selectable options for user interaction with the AI system. A timeline interface 1114 may appear at the bottom of the GUI 1100 showing a date indicator with various event markers and navigation controls. A text input field 1118 with a send button may be positioned below the suggestions in the AI interaction interface, allowing users to submit queries to the AI system.

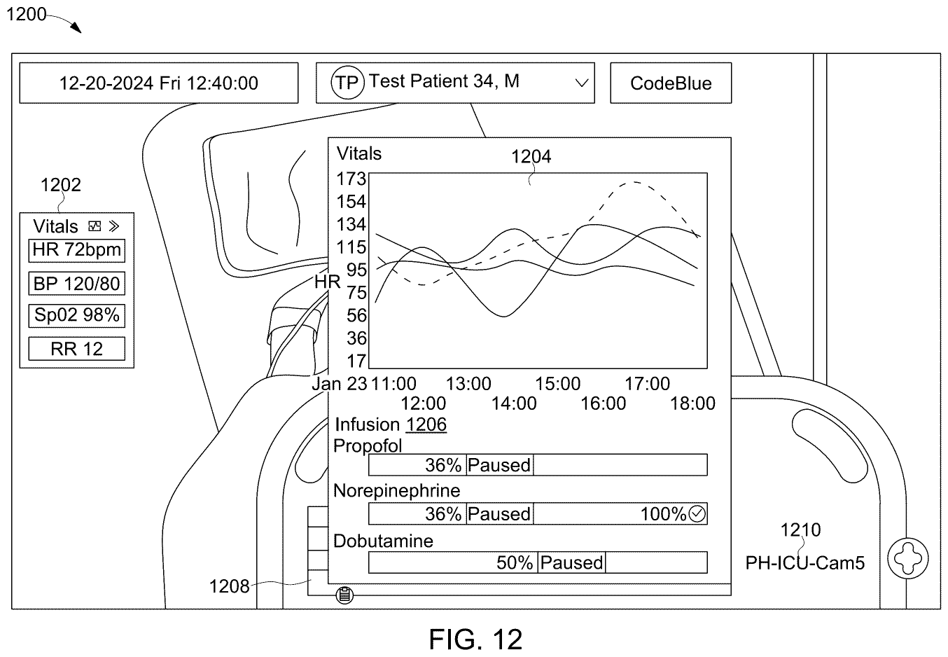

Referring now to FIG. 12, a GUI 1200 for interactive AI-based patient monitoring is illustrated, in accordance with some embodiments of the present disclosure. FIG. 12 is explained in conjunction with FIGS. 1-11. The GUI 1200 may display a real-time video feed of a patient in a hospital bed as the background, with the patient visible lying in the bed. A patient information bar may be shown at the top of the GUI 1200, displaying patient identification information including initials, name, age, and gender, along with an alert indicator. A vitals panel 1202 may be positioned on the left side of the GUI 1200, showing current patient vital signs including heart rate, blood pressure, SpO2, and respiratory rate. A vitals graph 1204 may be displayed in the center of the GUI 1200, showing multiple trend lines representing vital signs over time. An infusion panel 1206 may be shown below the vitals graph, displaying medication infusion status for various medications with progress indicators showing paused and active states. A timeline interface 1208 may be positioned at the bottom of the GUI 1200, allowing navigation through recorded video segments. A camera identifier 1210 may be displayed in the lower right portion of the GUI 1200, along with an AI interaction interface icon 1212.

As will be also appreciated, the above-described techniques may take the form of computer or controller implemented processes and apparatuses for practicing those processes. The disclosure can also be embodied in the form of computer program code containing instructions embodied in tangible media, such as floppy diskettes, solid state drives, CD-ROMs, hard drives, or any other computer-readable storage medium, wherein, when the computer program code is loaded into and executed by a computer or controller, the computer becomes an apparatus for practicing the invention. The disclosure may also be embodied in the form of computer program code or signal, for example, whether stored in a storage medium, loaded into and/or executed by a computer or controller, or transmitted over some transmission medium, such as over electrical wiring or cabling, through fiber optics, or via electromagnetic radiation, wherein, when the computer program code is loaded into and executed by a computer, the computer becomes an apparatus for practicing the invention. When implemented on a general-purpose microprocessor, the computer program code segments configure the microprocessor to create specific logic circuits.

The disclosed methods and systems may be implemented on a conventional or a general-purpose computer system, such as a personal computer (PC) or server computer. Referring now to FIG. 12, an exemplary computing system 1200 that may be employed to implement processing functionality for various embodiments (e.g., as a SIMD device, client device, server device, one or more processors, or the like) is illustrated. Those skilled in the relevant art will also recognize how to implement the invention using other computer systems or architectures. The computing system 1200 may represent, for example, a user device such as a desktop, a laptop, a mobile phone, personal entertainment device, DVR, and so on, or any other type of special or general-purpose computing device as may be desirable or appropriate for a given application or environment. The computing system 1200 may include one or more processors, such as a processor 1202 that may be implemented using a general or special purpose processing engine such as, for example, a microprocessor, microcontroller or other control logic. In this example, the processor 1202 is connected to a bus 1204 or other communication medium. In some embodiments, the processor 1202 may be an Artificial Intelligence (AI) processor, which may be implemented as a Tensor Processing Unit (TPU), or a graphical processor unit, or a custom programmable solution Field-Programmable Gate Array (FPGA).

The computing system 1200 may also include a memory 1206 (main memory), for example, Random Access Memory (RAM) or other dynamic memory, for storing information and instructions to be executed by the processor 1202. The memory 1206 also may be used for storing temporary variables or other intermediate information during execution of instructions to be executed by the processor 1202. The computing system 1200 may likewise include a read only memory (“ROM”) or other static storage device coupled to bus 1204 for storing static information and instructions for the processor 1202.

The computing system 1200 may also include storage devices 1208, which may include, for example, a media drive 1210 and a removable storage interface. The media drive 1210 may include a drive or other mechanism to support fixed or removable storage media, such as a hard disk drive, a floppy disk drive, a magnetic tape drive, an SD card port, a USB port, a micro-USB, an optical disk drive, a CD or DVD drive (R or RW), or other removable or fixed media drive. A storage media 1212 may include, for example, a hard disk, magnetic tape, flash drive, or other fixed or removable medium that is read by and written to by the media drive 1210. As these examples illustrate, the storage media 1212 may include a computer-readable storage medium having stored there in particular computer software or data.

In alternative embodiments, the storage devices 1208 may include other similar instrumentalities for allowing computer programs or other instructions or data to be loaded into the computing system 1200. Such instrumentalities may include, for example, a removable storage unit 1214 and a storage unit interface 1216, such as a program cartridge and cartridge interface, a removable memory (for example, a flash memory or other removable memory module) and memory slot, and other removable storage units and interfaces that allow software and data to be transferred from the removable storage unit 1214 to the computing system 1200.

The computing system 1200 may also include a communications interface 1218. The communications interface 1218 may be used to allow software and data to be transferred between the computing system 1200 and external devices. Examples of the communications interface 1218 may include a network interface (such as an Ethernet or other NIC card), a communications port (such as for example, a USB port, a micro-USB port), Near field Communication (NFC), etc. Software and data transferred via the communications interface 1218 are in the form of signals which may be electronic, electromagnetic, optical, or other signals capable of being received by the communications interface 1218. These signals are provided to the communications interface 1218 via a channel 1220. The channel 1220 may carry signals and may be implemented using a wireless medium, wire or cable, fiber optics, or other communications medium. Some examples of the channel 1220 may include a phone line, a cellular phone link, an RF link, a Bluetooth link, a network interface, a local or wide area network, and other communications channels.

The computing system 1200 may further include Input/Output (I/O) devices 1222. Examples may include, but are not limited to a display, keypad, microphone, audio speakers, vibrating motor, LED lights, etc. The I/O devices 1222 may receive input from a user and also display an output of the computation performed by the processor 1202. In this document, the terms “computer program product” and “computer-readable medium” may be used generally to refer to media such as, for example, the memory 1206, the storage devices 1208, the removable storage unit 1214, or signal(s) on the channel 1220. These and other forms of computer-readable media may be involved in providing one or more sequences of one or more instructions to the processor 1202 for execution. Such instructions, generally referred to as “computer program code” (which may be grouped in the form of computer programs or other groupings), when executed, enable the computing system 1200 to perform features or functions of embodiments of the present invention.

In an embodiment where the elements are implemented using software, the software may be stored in a computer-readable medium and loaded into the computing system 1200 using, for example, the removable storage unit 1214, the media drive 1210 or the communications interface 1218. The control logic (in this example, software instructions or computer program code), when executed by the processor 1202, causes the processor 1202 to perform the functions of the invention as described herein.

Various embodiments provide method and system for interactive AI-based patient monitoring. The disclosed method and system may receive in real-time, multimodal data of a patient through a set of sensors. The multimodal data may include at least one of image data, video data, or audio data. Further, the method and system may split the multimodal data into a set of chunks based on a predefined criterion. Each of the set of chunks may be mapped to a corresponding time interval of the multimodal data. Further, the method and system may assign, via a multimodal model, an index tag to each of the set of chunks. The index tag may include a textual description of a part of or whole of an associated chunk. Further, for each of the set of chunks, the method and system may determine, via a clinical decision model, a set of clinical scores or attributes for the patient during the corresponding time interval based on one or more keywords or one or more visual patterns identified in the textual description of the index tag or in a corresponding chunk, respectively. Further, the method and system may monitor, via the clinical decision model, the set of clinical scores or predictions of the patient at each time interval based on a corresponding set of predefined threshold scores or success criteria.

Thus, the disclosed techniques try to overcome the problem for interactive AI-based patient monitoring. The techniques may overcome the problem of delay in taking remedial actions in case of undesired vitals of the patient. The techniques may provide better care and supervision to the patient. The techniques may further incorporate easier access of the doctor through an AV interface. The techniques may further include easier user query resolution via an AI interface. The techniques may further keep track of improvement or deterioration of the health of the patient through the EMR data. The techniques may further enable automated clinical score calculation based on visual patterns and textual descriptions extracted from multimodal data, thereby reducing manual assessment burden on healthcare providers. The techniques may further facilitate timely alert generation when clinical scores exceed predefined threshold values, enabling proactive intervention before patient conditions deteriorate significantly.

In light of the above-mentioned advantages and the technical advancements provided by the disclosed method and system, the claimed steps as discussed above are not routine, conventional, or well understood in the art, as the claimed steps enable the following solutions to the existing problems in conventional technologies. Further, the claimed steps clearly bring an improvement in the functioning of the device itself as the claimed steps provide a technical solution to a technical problem.

The specification has a described method and system for interactive AI-based patient monitoring. The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., be non-transitory. Examples include random access memory (RAM), read-only memory (ROM), volatile memory, non-volatile memory, hard drives, CD ROMs, DVDs, flash drives, disks, and any other known physical storage media.

It is intended that the disclosure and examples be considered as exemplary only, with a true scope and spirit of disclosed embodiments being indicated by the following claims.

What is claimed is:

1. A method for interactive Artificial Intelligence (AI)-based patient monitoring, the method comprising:
receiving in real-time, by a processor, multimodal data of a patient through a set of sensors, wherein the multimodal data comprises at least one of image data, video data, or audio data;
splitting, by the processor, the multimodal data into a set of chunks based on a predefined criterion, wherein each of the set of chunks is mapped to a corresponding time interval of the multimodal data;
assigning, by the processor via a multimodal model, an index tag to each of the set of chunks, wherein the index tag comprises a textual description of a part or whole of an associated chunk;
for each of the set of chunks, determining, by the processor via a clinical decision model, a set of clinical scores or attributes for the patient during the corresponding time interval based on one or more keywords or one or more visual patterns identified in the textual description of the index tag or in a corresponding chunk, respectively; and
monitoring, by the processor via the clinical decision model, the set of clinical scores or predictions of the patient at each time interval based on a corresponding set of predefined threshold scores or success criteria.

2. The method of claim 1, wherein the predefined criterion corresponds to one of a predefined time interval or a predetermined content portion.

3. The method of claim 1, wherein assigning the index tag comprises:
for each chunk of the set of chunks:
extracting, via the multimodal model, relevant clinical information from the chunk;
generating, via the multimodal model, the textual description corresponding to the chunk; and
creating, via the multimodal model, the index tag for the chunk based on the clinical information and the textual description.

4. The method of claim 1, further comprising storing a mapping of the set of chunks and an associated set of index tags in a vector database.

5. The method of claim 4, wherein determining the set of clinical scores comprises:
for each chunk of the set of chunks,
retrieving the mapping of the chunk and an associated index tag from the vector database;
identifying, via the clinical decision model, one or more patterns in one of the textual description of the index tag or the chunk, wherein the one or more patterns comprises:
one or more keywords in the textual description of the index tag; or
one or more visual patterns in the chunk; and
determining, via the clinical decision model, the set of clinical scores or attributes based on the one or more patterns.

6. The method of claim 1, wherein monitoring the set of clinical scores of the patient comprises:
for each clinical score of the set of clinical scores or attributes,
comparing, via the clinical decision model, the clinical score with a corresponding predefined threshold score; and
rendering, via a Graphical User Interface (GUI), an alert corresponding to the patient based on the comparing.

7. The method of claim 6, further comprising:
generating, via the clinical decision model, a clinical practice recommendation corresponding to the patient based on the monitoring of the set of clinical scores or attributes, in response to a user query; and
rendering, via the GUI, the clinical practice recommendation corresponding to the patient, wherein the clinical practice recommendation is overlaid on a real-time video of the patient in the GUI.

8. A system for interactive Artificial Intelligence (AI)-based patient monitoring, the system comprising:
a processor; and
a memory communicatively coupled to the processor, wherein the memory stores processor instructions, which when executed by the processor, cause the processor to:
receive in real-time, multimodal data of a patient through a set of sensors, wherein the multimodal data comprises at least one of image data, video data, or audio data;
split the multimodal data into a set of chunks based on a predefined criterion, wherein each of the set of chunks is mapped to a corresponding time interval of the multimodal data;
assign, via a multimodal model, an index tag to each of the set of chunks, wherein the index tag comprises a textual description of a part or whole of an associated chunk;
for each of the set of chunks, determine, via a clinical decision model, a set of clinical scores or attributes for the patient during the corresponding time interval based on one or more keywords or one or more visual patterns identified in the textual description of the index tag or in a corresponding chunk, respectively; and
monitor, via the clinical decision model, the set of clinical scores or predictions of the patient at each time interval based on a corresponding set of predefined threshold scores or success criteria.

9. The system of claim 8, wherein the predefined criterion corresponds to one of a predefined time interval or a predetermined content portion.

10. The system of claim 8, wherein to assign the index tag, the processor instructions, on execution, cause the processor to:
for each chunk of the set of chunks:
extract, via the multimodal model, relevant clinical information from the chunk;
generate, via the multimodal model, the textual description corresponding to the chunk; and
create, via the multimodal model, the index tag for the chunk based on the clinical information and the textual description.

11. The system of claim 8, wherein the processor instructions, on execution, further cause the processor to store a map of the set of chunks and an associated set of index tags in a vector database.

12. The method of claim 11, wherein to determine the set of clinical scores, the processor instructions, on execution, cause the processor to:
for each chunk of the set of chunks,
retrieve the mapping of the chunk and an associated index tag from the vector database; and
identify, via the clinical decision model, one or more patterns in one of the textual description of the index tag or the chunk, wherein the one or more patterns comprises:
one or more keywords in the textual description of the index tag; or
one or more visual patterns in the chunk; and
determine, via the clinical decision model, the set of clinical scores or attributes based on the one or more patterns.

13. The system of claim 8, wherein to monitor the set of clinical scores of the patient, the processor instructions, on execution, cause the processor to:
for each clinical score of the set of clinical scores or attributes,
compare, via the clinical decision model, the clinical score with a corresponding predefined threshold score; and
render, via a GUI, an alert corresponding to the patient based on the comparing.

14. The system of claim 13, wherein the processor instructions, on execution, further cause the processor to:
generate, via the clinical decision model, a clinical practice recommendation corresponding to the patient based on the monitoring of the set of clinical scores or attributes, in response to a user query; and
render, via the GUI, the clinical practice recommendation corresponding to the patient, wherein the clinical practice recommendation is overlaid on a real-time video of the patient in the GUI.

15. A non-transitory computer-readable medium storing computer-executable instructions for interactive Artificial Intelligence (AI)-based patient monitoring, the computer-executable instructions configured for:
receiving in real-time, multimodal data of a patient through a set of sensors, wherein the multimodal data comprises at least one of image data, video data, or audio data;
splitting the multimodal data into a set of chunks based on a predefined criterion, wherein each of the set of chunks is mapped to a corresponding time interval of the multimodal data;
assigning, via a multimodal model, an index tag to each of the set of chunks, wherein the index tag comprises a textual description of a part or whole of an associated chunk;
for each of the set of chunks, determining, via a clinical decision model, a set of clinical scores or attributes for the patient during the corresponding time interval based on one or more keywords or one or more visual patterns identified in the textual description of the index tag or in a corresponding chunk, respectively; and
monitoring, via the clinical decision model, the set of clinical scores or predictions of the patient at each time interval based on a corresponding set of predefined threshold scores or success criteria.

16. The non-transitory computer-readable medium of claim 15, wherein the predefined criterion corresponds to one of a predefined time interval or a predetermined content portion.

17. The non-transitory computer-readable medium of claim 15, wherein for assigning the index tag, the computer-executable instructions are configured for:
for each chunk of the set of chunks:
extracting, via the multimodal model, relevant clinical information from the chunk;
generating, via the multimodal model, the textual description corresponding to the chunk; and
creating, via the multimodal model, the index tag for the chunk based on the clinical information and the textual description.

18. The non-transitory computer-readable medium of claim 15, wherein the computer-executable instructions are further configured for storing a mapping of the set of chunks and an associated set of index tags in a vector database.

19. The non-transitory computer-readable medium of claim 18, wherein for determining the set of clinical scores, the computer-executable instructions are configured for:
for each chunk of the set of chunks,
retrieving the mapping of the chunk and an associated index tag from the vector database;
identifying, via the clinical decision model, one or more patterns in one of the textual description of the index tag or the chunk, wherein the one or more patterns comprises:
one or more keywords in the textual description of the index tag; or
one or more visual patterns in the chunk; and
determining, via the clinical decision model, the set of clinical scores or attributes based on the one or more patterns.

20. The non-transitory computer-readable medium of claim 15, wherein for monitoring the set of clinical scores of the patient, the computer-executable instructions are configured for:
for each clinical score of the set of clinical scores or attributes,
comparing, via the clinical decision model, the clinical score with a corresponding predefined threshold score; and
rendering, via a Graphical User Interface (GUI), an alert corresponding to the patient based on the comparing.
